SEQUENCE LISTING (48 sequences)

SEQ ID NO: 1 - protein, 580 aa, Synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNKESTQIIIIIIVCILIIIICGILYYLYRVRRLLIMINSTNNSPINAYTLESRMKNPYMGNHSNKIRSSTLHTYNNQIYPQLLSDVVRK

SEQ ID NO: 2 - protein, 467 aa, Synthetic construct
QIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVG

SEQ ID NO: 3 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGSGEPEA

SEQ ID NO: 4 - protein, 494 aa, Synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 5 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 6 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNKLEEVKTELMKVRAIISAVGSGEPEA

SEQ ID NO: 7 - protein, 494 aa, Synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 8 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGPGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 9 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIPEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 10 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 11 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 12 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQPIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 13 - protein, 494 aa, Synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 14 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAKISAVGSGEPEA

SEQ ID NO: 15 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELKKVRAIISAVGSGEPEA

SEQ ID NO: 16 - protein, 494 aa, Synthetic construct
MQSSEILILAYSSLLLSSSLCQIPVDKLSNVGVIINEPKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 17 - protein, 494 aa, Synthetic construct
MQSSEILILAYSSLLLSSSLCQIPVDKLSNVGVIINEGKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 18 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 19 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 20 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIAIKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 21 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFGSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 22 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 23 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIFTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 24 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 25 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 26 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 27 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFGSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 28 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 29 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIFTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 30 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIAIKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 31 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFGSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 32 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFSSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 33 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFGSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 34 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 35 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIFTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 36 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIAIKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 37 - protein, 494 aa, Synthetic construct
MQSSEILILAYSSLLLSSSLCQIPVDKLSNVGVIINEPKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 38 - protein, 494 aa, Synthetic construct
MQSSEILILAYSSLLLSSSLCQIPVDKLSNVGVIINEGKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 39 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 40 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIAIKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 41 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFGSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 42 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 43 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIFTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 44 - protein, 494 aa, Synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 45 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 46 - protein, 494 aa, Synthetic construct; identical to SEQ ID NO: 6

SEQ ID NO: 47 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIALKTLQDFVNDEIRPAIGELRCETTALKLGIKLTQHYSELATAFGSNLGTIGEKGLTLQALSSLYSANITEILSTIKKDKSDIYDIIYTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 48 - protein, 494 aa, Synthetic construct
MQSSEILILAYSSLLLSSSLCQIPVDKLSNVGVIINEPKGLKIAGSYESRYIVLSLVPSIDLQDGCGTTQIIQYKNLLNRLLIPLKDALDLQESLITITNDTTVTNDNPQTRFFGAVIGTIALGVATAAQITAAIALAEAREARKDIALIKDSIVKTHNSVEFIQRGIGEPIIAIKTLQDFVNDEIRPAIGELRCETTALKLGIKLTQHYSELATAFGSNLGTIGEKGLTLQALSSLYSANITEILSTIKKDKSDIYDIIFTEQVKGTVIDVDLEKYMVTLLVKIPILSEIPGVLIYRASSISYNIEGEEWHVAIPNYIISKGSSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

(19) United States
(12) Patent Application Publication
(10) Pub. No.: US 2026/0139011 A1
(43) Pub. Date: May 21, 2026

(54) PRE-FUSION HUMAN PIV1 F PROTEINS

(71) Applicant: JANSSEN VACCINES & PREVENTION B.V., Leiden (NL)

(72) Inventors: Johannes Petrus Maria LANGEDIJK, Amsterdam (NL); Mark Johannes Gerardus BAKKERS, Haarsteeg (NL)

(21) Appl. No.: 19/113,802

(22) PCT Filed: Sep. 15, 2023
(86) PCT No.: PCT/EP2023/075405

§ 371 Date: Mar. 20, 2025

(30) Foreign Application Priority Data
Sep. 23, 2022 (EP) 22197347.2

(51) Int. Cl. C07K 14/115; A61K 39/155; C12N 7/02

(52) U.S. Cl. CPC C07K 14/115; A61K 39/155; C12N 7/02; C12N 2760/18622; C12N 2760/18634; C12N 2760/18651

(57) ABSTRACT

The present invention relates to stabilized human parainfluenza virus 1 (HPIV1) F protein, and to nucleic acid sequences encoding such proteins, as well as to uses of said proteins and nucleic acid sequences.

The present invention relates to the field of medicine. The invention, in particular, relates to recombinant human PIV1 F (HPIV1) proteins, and to fragments thereof, to nucleic acid molecules encoding the HPIV1 F proteins and fragments, and to uses thereof, e.g. in vaccines.

### BACKGROUND OF THE INVENTION

Human parainfluenza virus (HPIV) induces respiratory complications mainly in children and the immunocompromised; however, more recently, it has been identified as a concern in the adult population as well. HPIV infection is associated with 7600 to 48,000 pediatric hospitalizations per year in the US and is an important cause of mortality, morbidity, and health care costs in other vulnerable populations. Most children have experienced an HPIV infection by 5 years of age, and by adulthood, more than 90% of humans have antibodies against HPIV (Ison et al. (2019) Clinical Microbiology Reviews, 32: e00042-19).

There is currently no vaccine and no specific antiviral treatment to prevent HPIV illness. Medical care is supportive, except for croup where the use of corticosteroids and nebulized epinephrine has been found to be beneficial.

Four serotypes of HPIV are known (HPIV1 through-4), which are associated with distinct clinical presentations and seasonal incidence, with HPIV1 causing seasonal outbreaks (typically in the fall of odd-numbered years) and commonly presenting as croup in children. Seasonal variations in the different serotypes and spontaneous outbreaks drive an overall variable incidence and complex epidemiology (Ison et al. (2019) Clin Microbiol Rev, 32: e00042-19).

HPIV1 is an enveloped RNA virus in the Paramyxoviridae family of the order Mononegavirales. It has a genome of ˜15,000 nucleotides in length that encodes six key proteins in the following gene sequence: 3′-N-P-M-F-HN-L-5 (Rima et al. (2019), J Gen Virol, 100:1593-1594).

Fusion of viral and host cell membranes results from the coordinated action of the two envelope glycoproteins that comprise the viral entry machinery: a receptor binding protein, hemagglutinin-neuraminidase (HN), and a fusion protein (F). Upon binding to sialic acid-containing target receptors, HN, a molecule with both receptor binding and cleaving activities, triggers and activates the F protein. The F protein fuses the viral and host-cell membranes by irreversible protein refolding from the labile pre-fusion (preF) conformation to the stable post-fusion (postF) conformation. Structures of both conformations have been determined for several paramyxoviruses, providing insight into the complex mechanism of this fusion protein (Stewart-Jones et al. (2018), Proc Natl Acad Sci USA, 115:12265-12270; Yin et al. (2005), Proc Natl Acad Sci USA, 102:9288-9293; Yin et al. (2006), Nature, 439:38-44; Swanson et al. (2010), Virology, 402:372-379; Wong et al. (2016), Proc Natl Acad Sci USA, 113:1056-1061.)

As a type I transmembrane protein, F is translated at the endoplasmic reticulum and transported through the Golgi apparatus and trans-Golgi network to the plasma membrane. Similar to other class I fusion proteins, the inactive precursor, HPIV1 F₀, requires cleavage into the disulfide-linked subunits F1 and F2 by appropriate host endoproteases, likely TMPRSS2, at a monobasic cleavage site (Abe et al. (2013), J Virol, 87:11930-11935). After this cleavage, F1 contains a hydrophobic fusion peptide (FP) at its N-terminus. In order to refold from the pre-fusion to the post-fusion conformation, the refolding region 1 (RR1) between residue 113 and 214, that includes the FP and heptad repeat A (HRA, also referred to as ‘HR1’), wherein the numbering is based on the numbering of amino acid residues in SEQ ID NO: 1), has to transform from an assembly of helices, loops, and beta-strands to a long continuous helix. The FP, located at the N-terminal segment of RR1, is then able to extend away from the viral membrane and to insert into the proximal membrane of the target cell. Next, the refolding region 2 (RR2, comprising amino acid residues 432-487), which forms the C-terminal stem in the pre-fusion F spike and includes the heptad repeat B (HRB, also referred to as ‘HR2’), relocates to the other side of the HPIV1 F head and binds the extended HRA coiled-coil trimer with the HRB domain to form the six-helix bundle. The formation of the RRI coiled-coil and relocation of RR2 to complete the six-helix bundle are the most dramatic structural changes that occur during the refolding process (Welch et al. (2012), Proc Natl Acad Sci USA, 109:16672-16677).

Class I fusion proteins have been shown to be inherently unstable. Structure-based stabilization of viral fusion protein in the pre-fusion conformation has been shown to induce superior neutralization and protection in animal models and clinical trials (Krarup et al. (2015) Nat Commun, 6:8143; De Taeye et al. (2015), Cell, 163:1702-1715; Mclellan et al. (2013), Science, 342:592-598; Stewart-Jones et al. (2018). Proc Natl Acad Sci USA, 48:12265-12270; Crank et al., (2019), Science, 365:505-509; Sadoff et al. (2021), J Infect Dis, doi: 10.1093/infdis/jiab003; Sadoff et al. (2021), N Engl J Med, 384; 2187-2201), but until this date, still no vaccine is available for prevention of HPIV1.

A need remains for efficient vaccines against HPIV1, in particular vaccines comprising or based on HPIV1 F proteins stabilized in the pre-fusion conformation. Indeed, vaccines, preferably indicated for pediatric and high-risk patients (e.g., elderly and COPD patients) could provide broad impact intervention, preventing serious illness thereby reducing HPIV1 overall incidence and associated morbidity and mortality. The present invention aims at providing means for obtaining stable HPIV1 F protein, e.g. for use in vaccinating against HPIV1.

### SUMMARY OF THE INVENTION

The present invention provides stable, recombinant, human parainfluenza type I (HPIV1) fusion (F) proteins, i.e. recombinant HPIV1 F proteins that are stabilized in the trimeric, pre-fusion conformation, and fragments thereof. The invention also provides nucleic acid molecules encoding the trimeric HPIV1 F proteins, or fragments thereof, as well as vectors, e.g. adenovectors, comprising such nucleic acid molecules.

The invention further relates to compositions, preferably pharmaceutical compositions, comprising an HPIV1 F protein, a nucleic acid molecule and/or a vector, as described herein, and to the use thereof in inducing an immune response against HPIV1 F protein, in particular to the use thereof as a vaccine against HPIV1. The invention also relates to methods for inducing an anti-HPIV1 immune response in a subject, comprising administering to the subject an effective amount of a trimeric HPIV1 F protein, a nucleic acid molecule encoding said HPIV1 F protein, and/or a vector comprising said nucleic acid molecule, as described herein. Preferably, the induced immune response is characterized by the induction of neutralizing antibodies to HPIV1 and/or protective immunity against HPIV1. In particular aspects, the invention relates to a method for inducing anti-HPIV1 F antibodies in a subject, comprising administering to the subject an effective amount of a pharmaceutical composition comprising a trimeric HPIV1 F protein, a nucleic acid molecule encoding said HPIV1 F protein, and/or a vector comprising said nucleic acid molecule, as described herein.

The invention also relates to methods of stabilizing HPIV1 F proteins in the trimeric conformation, and to the trimeric HPIV1 F proteins obtainable by said methods.

### BRIEF DESCRIPTION OF THE FIGURES

The foregoing summary, as well as the following detailed description of the invention, will be better understood when read in conjunction with the appended figures. It should be understood that the invention is not limited to the precise embodiments shown in the examples.

FIG. 1: Schematic representation of the conserved elements of the HPIV1 F protein in both the full-length, membrane bound protein (‘full-length’, top panel) and in the mature, soluble ectodomain (‘ectodomain’, bottom panel). The N-terminal F2 domain is preceded by a signal peptide sequence (SP) that is cleaved off during protein maturation. The fusion peptide (FP) is located at the N-terminus of F1. Heptad repeats A, B and C are indicated (HRA (HR1), HRB (HR2), HRC, respectively). Further indicated are the transmembrane region (TM) and cytoplasmic tail (CT). Cleavage site between SP and F2 and between F2 and F1 are indicated with arrows.

FIG. 2: Analytical SEC profiles of HPIV1 F proteins with stabilizing mutations in crude cell supernatant.

HPIV1 F trimer detection in supernatant of cells transfected with HPIV1 F constructs using analytical size exclusion chromatography (SEC). The peak between 4.4- and 4.8-minutes retention time corresponds to the HPIV1 F trimer.

FIG. 3: Analytical SEC profiles of stabilized HPIV1 F proteins based on PIV211400 in crude cell supernatant.

HPIV1 F trimer detection in supernatant of cells transfected with HPIV1 F constructs using analytical SEC. The peak at approximately 4.6 minutes retention time corresponds to the HPIV1 F trimer.

FIG. 4: Analytical SEC and melting temperature of stabilized HPIV1 F proteins based on PIV211843 in crude cell supernatant.

(A) HPIV1 F trimer detection in supernatant of cells transfected with HPIV1 F constructs using analytical SEC. The peak at approximately 4.5 minutes retention time corresponds to the HPIV1 F trimer.

(B) Melting temperature of HPIV1 F trimer in supernatant of transfected cells from (A) as determined by differential scanning fluorimetry (DSF). The average of n=3 replicate measurements is reported with error bar indicating the standard deviation. The dashed line represents the average melting point of backbone PIV211843.

FIG. 5: Analytical SEC and melting temperature of stabilized HPIV1 F proteins based on PIV211847 in crude cell supernatant.

(A) HPIV1 F trimer detection in supernatant of cells transfected with HPIV1 F constructs using analytical SEC. The peak at approximately 4.5 minutes retention time corresponds to the HPIV1 F trimer.

(B) Melting temperature of HPIV1 F trimer in supernatant of transfected cells from (A) as determined by DSF. The average of n=3 replicate measurements is reported with error bar indicating the standard deviation. The dashed line represents the average melting point of backbone PIV211847.

FIG. 6: Purification and characterization of differently stabilized HPIV1 F trimers without a heterologous trimerization domain.

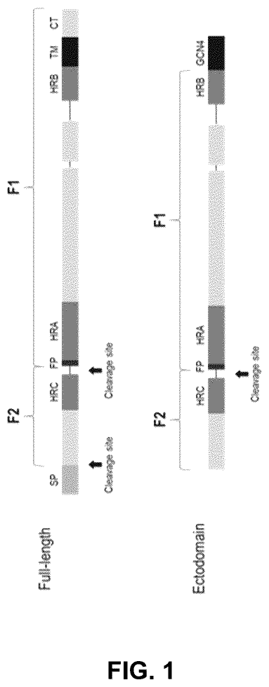

(A) Characterization of purified HPIV1 F trimers without a heterologous trimerization domain and with multiple amino acid substitutions as indicated in the table. The mass throughout the trimer peak as determined by MALS is shown as a gray line.

(B) Melting temperature of purified HPIV1 F trimer of (A) as determined by DSF. n=3 replicate measurements, and individual and average values are reported as grey and black solid lines, respectively.

(C) Negative stain electron microscopy (EM) of purified PIV220147 HPIV1 F trimer from (A) with representative two-dimensional (2D) class averages.

### DETAILED DESCRIPTION OF THE INVENTION

Human parainfluenza virus type 1 (HPIV1) is a significant cause of severe respiratory tract disease in infants and young children. HPIV1 is an enveloped, non-segmented, single-stranded, negative-sense RNA virus belonging to the subfamily Paramyxovirinae within the Paramyxoviridae family, which also includes the HPIV2, HPIV1 and HPIV4 serotypes. These serotypes can be further classified as belonging to either the Respirovirus (HPIV1 and HPIV1) or Rubulavirus (HPIV2 and HPIV4) genus and are immunologically distinct in that primary infection does not result in cross-neutralization or cross-protection. HPIVs cause respiratory tract disease ranging from mild illness, including rhinitis, pharyngitis, and otitis media, to severe disease, including croup, bronchiolitis, and pneumonia. A licensed vaccine is currently not available for any of the HPIVs.

The present invention provides human parainfluenza virus 1 (HPIV1) F proteins, comprising an F1 and an F2 domain, or fragments thereof, comprising an amino acid sequence of the F1 and F2 domain of an F protein of an HPIV1 strain, or fragments thereof, comprising an hydrophobic amino acid at position 473 and at position 480, and wherein the amino acid residue at position 171 is P, the amino acid residue at position 44 is P, the amino acid residue at position 134 is A, the amino acid residue at position 175 is I, the amino acid residue at position 218 is G, the amino acid residue at position 469 is K, the amino acid residue at position 168 is P, the amino acid residue at position 170 is P, the amino acid residue at position 38 P, and/or the amino acid residue at position 40 is G, and/or the amino acid at position 38 is P and the amino acid residue at position 40 is G, wherein the numbering of the amino acid positions is according to the numbering of the amino acid residues in SEQ ID NO: 1.

The present invention provides stabilized trimeric pre-fusion HPIV1 proteins that show high expression levels and increased stability.

According to the invention it has been demonstrated that the presence of one or more of the specific amino acid residues at the indicated positions increases the stability of the HPIV1 F proteins and/or HPIV1 F protein ectodomains in the pre-fusion conformation, as compared to HPIV1 F protein without these amino acid residues at these positions. According to the invention, the specific amino acids can be either already present in the amino acid sequence or can be introduced by substitution (mutation) of the amino acid on that position into the specific amino acid according to the invention.

It is noted that the terms HPIV1 and PIV1 are used interchangeably throughout this application.

In certain embodiments, the proteins or fragments, comprise a hydrophobic amino acid at position 473 and at position 480, and the amino acid residue at position 171 is P.

In certain embodiments, the proteins or fragments, comprise a hydrophobic amino acid at position 473 and at position 480, and the amino acid residue at position 171 is P, and furthermore the amino acid residue at position 38 is P, the amino acid at position 40 is G, the amino acid residue at position 44 is P, the amino acid residue at position 134 is A, the amino acid residue at position 175 is I, the amino acid at position 218 is G, the amino acid residue at position 228 is G, the amino acid residue at position 261 is F, the amino acid residue at position 478 is K, the amino acid residue at position 483 is K and/or the amino acid residue at position 323 is G, and/or the amino acid at position 38 is P and the amino acid residue at position 40 is G.

In a preferred embodiment, the proteins comprise a hydrophobic amino acid at position 473 and at position 480, the amino acid residue at position 171 is P and the amino acid at position 38 is P and the amino acid at position 40 is G.

In another embodiment, the proteins comprise a hydrophobic amino acid at position 473 and at position 480, the amino acid residue at position 171 is P and the amino acid at position 38 is P and the amino acid at position 40 is G, and furthermore the amino acid residue at position 134 is A, the amino acid residue at position 175 is I, the amino acid residue at position 218 is G, the amino acid residue at position 228 is G, the amino acid residue at position 261 is F and/or the amino acid residue at position 323 is G.

In certain other embodiments, the proteins comprise a hydrophobic amino acid at position 473 and at position 480, and the amino acid residue at position 171 is P and the amino acid residue at position 134 is A.

In further embodiments, the proteins comprise a hydrophobic amino acid at position 473 and at position 480, and the amino acid residue at position 171 is P and the amino acid residue at position 134 is A, and furthermore the amino acid residue at position 38 is P and the amino acid residue at position 40 is G, and/or the amino acid residue at position 175 is I, the amino acid residue at position 218 is G, the amino acid residue at position 261 is F, the amino acid residue at position 228 is G, and/or the amino acid residue at position A323 is G.

In a preferred embodiment, the proteins comprise a hydrophobic amino acid at position 473 and at position 480, and the amino acid residue at position 171 is P, the amino acid residue at position 170 is P and the amino acid residue at position 44 is P.

In other preferred embodiments, the proteins comprise a hydrophobic amino acid at position 473 and at position 480, the amino acid residue at position 171 is P, the amino acid at position 38 is P, the amino acid residue at position 40 is G, the amino acid residue 134 is A, the amino acid residue at position 218 is G and the amino acid residue at position 228 is G.

According to the invention, the hydrophobic amino acid at positions 473 and/or 480 is selected from the group consisting of valine (V), leucine (L), isoleucine (I), and methionine (M). The amino acid residues at position 473 and 480 may be the same hydrophobic amino acid, or different hydrophobic amino acids. In certain preferred embodiments, the hydrophobic amino acid at position 473 and/or 480 is valine (V), preferably both the amino acid at position 473 and 480 are valine (V).

In certain embodiments, the proteins have an increased stability (thermostability) upon storage a 4° C., and/or at 50° C. and/or or 60° C., as compared to HPIV1 F proteins without the presence of these amino acid residues at these positions. With “stability upon storage”, it is meant that the proteins are still trimeric upon storage of the protein in solution (e.g. culture medium) at 4°, 50° C. and/or or 60° C. for a predetermined period of time.

In addition, or alternatively, the proteins may have an increased thermostability, e.g. as indicated by an increased melting temperature (measured by e.g. differential scanning fluorimetry).

The invention also provides fragments of the HPIV1 F proteins. The term “fragment” as used herein refers to a HPIV1 polypeptide that has an amino-terminal (e.g. by cleaving off the signal sequence) and/or carboxy-terminal (e.g. by deleting the transmembrane region and/or cytoplasmic tail) and/or internal deletion, but wherein the remaining amino acid sequence is identical to the corresponding positions in the sequence of the HPIV1 F protein, for example, the full-length sequence of a HPIV1 F protein. It will be appreciated that for inducing an immune response and in general for vaccination purposes, a protein needs not to be full length nor have all its wild type functions, and fragments of the protein are equally useful. A fragment according to the invention is an immunologically active fragment, and typically comprises at least 15 amino acids, or at least 30 amino acids of the HPIV1 F protein. In certain embodiments, a fragment comprises at least 50, 75, 100, 150, 200, 250, 300, 350, 400, 450, 460, 470, 480, 490, 500, or 510 amino acids of the HPIV13 F protein. In a preferred embodiment, the fragment is an HPIV1 F protein ectodomain, consisting of the amino acid residues 22-487 of the HPIV1 F protein.

In certain embodiments, the proteins or fragments thereof according to the invention do not comprise a signal sequence. It will be understood by the skilled person that signal sequences (sometimes referred to as signal peptide, targeting signal, localization signal, localization sequence, transit peptide, leader sequence or leader peptide) function to prompt a cell to translocate the protein, usually to the cellular membrane. Signal peptidase may cleave either during or after completion of translocation to generate a free signal peptide and a mature protein.

In certain embodiments, the PIV1 F protein ectodomain comprises a truncated F1 domain, preferably the truncated F1 domain does not comprise the transmembrane and cytoplasmic regions of the HPIV1 F protein. According to the invention said truncated F1 domain may comprise the amino acids 113-488, preferably the amino acids 113-488. In certain embodiments, the truncates F1 domain consists of the amino acids 113-488, preferably the amino acids 113-488 of the HPIV1 F protein.

In order to promote stable trimerization of the HPIV1 F ectodomains, a heterologous trimerization domain may be linked to the truncated F1 domain.

As described above, because the TM region is responsible for membrane anchoring and increases stability, the ectodomain of the F protein is considerably more labile than the full-length protein and will even more readily refold into the post-fusion end-state. In order to obtain stable soluble F protein in the pre-fusion conformation that shows high expression levels and high stability in certain embodiments a heterologous trimerization domain may be linked to the truncated F1 domain. The heterologous trimerization domain can be a GCN4 Leucine-Zipper domain. According to the invention, the heterologous trimerization domain may comprise, or consist of, the amino acid sequence of SEQ ID NO: 3. Alternative versions of GCN4 domains, or other heterologous trimerizations domains are also suitable according to the invention.

As used throughout the present application, the amino acid positions are given in reference to a wild type sequence of the HPIV1 F protein of SEQ ID NO: 1. As used in the present invention, the wording “the amino acid residue at position “x” of the F protein thus means the amino acid residue corresponding to the amino acid residue at position “x” in the HPIV1 F protein of SEQ ID NO: 1. Note that, in the numbering system used throughout this application 1 refers to the N-terminal amino acid of an immature F0 protein (SEQ ID NO: 1). When an F protein of another HPIV1 strain is used, the amino acid positions of the F protein are to be numbered with reference to the numbering of the F protein of SEQ ID NO: 1 by aligning the sequences of the other HPIV1 F protein with the F protein of SEQ ID NO: 1 with the insertion of gaps as needed. Sequence alignments can be done using methods well known in the art, e.g. by CLUSTALW, Bioedit or CLC Workbench.

In certain preferred embodiments, the proteins are trimeric and do not comprise a heterologous trimerization domain.

The present invention in particular provides soluble trimeric human parainfluenza virus 1 (HPIV1) F proteins, comprising a truncated F1 domain and an F2 domain, comprising an amino acid sequence of the truncated F1 and F2 domain of an F protein of an HPIV1 strain, wherein the amino acid residue at position 473 and/or 480 is a hydrophobic amino acid, and wherein the amino acid residue at position 171 is P, wherein the numbering of the amino acid positions is according to the numbering is amino acid residues in SEQ ID NO: 1, wherein the proteins do not comprise a heterologous trimerization domain.

According to the present invention, it has been demonstrated that stable soluble trimeric pre-fusion PIV1 ectodomains (i.e. soluble trimeric pre-fusion PIV1 proteins) can be obtained without the presence of a heterologous trimerization domain, when the amino acid residue at position 473 and/or the amino acid residue at position 480 is a hydrophobic amino acid, preferably when the amino acid residues at both position 473 and 480 are hydrophobic.

In certain embodiments, the truncated F1 domain does not comprise the transmembrane and cytoplasmic regions. Preferably, the truncated F1 domain comprises the amino acids 113-488. In certain embodiment, the truncated F1 domain consists of the amino acids 113-488 of the HPIV1 F protein.

In certain embodiments, the proteins comprise an amino acid sequence selected from the group consisting of SEQ ID NO: 4, 6-11, 13-36, 39-41, 45-48, or an amino acid sequence having at least 90%, preferably at least 95%, more preferably at least 97%, more preferably at least 99% amino acid sequence identity or a fragment thereof. Preferably, the protein comprises an amino acid sequence selected from the group consisting of SEQ ID NO: 31, SEQ ID NO: 47 and SEQ ID NO: 48, or a fragment thereof.

In certain embodiments, the proteins do not comprise a signal sequence.

In certain embodiments, the proteins do not comprise a C-terminal tag (C-tag).

As used throughout the present application nucleotide sequences are provided from 5′ to 3′ direction, and amino acid sequences from N-terminus to C-terminus, as custom in the art.

An amino acid according to the invention can be any of the twenty naturally occurring (or ‘standard’ amino acids). The standard amino acids can be divided into several groups based on their properties. Important factors are charge, hydrophilicity or hydrophobicity, size and functional groups. These properties are important for protein structure and protein-protein interactions. Some amino acids have special properties such as cysteine, that can form covalent disulfide bonds (or disulfide bridges) to other cysteine residues, proline that induces turns of the protein backbone, and glycine that is more flexible than other amino acids. Table 1 shows the abbreviations and properties of the standard amino acids.

It will be appreciated by a skilled person that the mutations can be made to the protein by routine molecular biology procedures. The mutations according to the invention preferably result in increased expression levels and/or increased stabilization of the pre-fusion PIV1 F proteins as compared to PIV1 F proteins that do not comprise these mutation(s).

The present invention further provides nucleic acid molecules encoding the PIV1 F proteins according to the invention.

In preferred embodiments, the nucleic acid molecules encoding the proteins according to the invention are codon-optimized for expression in mammalian cells, preferably human cells. Methods of codon-optimization are known and have been described previously (e.g. WO 96/09378). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

It will be understood by a skilled person that numerous different polynucleotides and nucleic acid molecules can encode the same protein as a result of the degeneracy of the genetic code. It is also understood that skilled persons may, using routine techniques, make nucleotide substitutions that do not affect the protein sequence encoded by the nucleic acid molecules to reflect the codon usage of any particular host organism in which the proteins are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may or may not include introns.

Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScripts, Invitrogen, Eurofins).

The invention also provides vectors comprising a nucleic acid molecule as described above. In certain embodiments, a nucleic acid molecule according to the invention thus is part of a vector.

In certain embodiments of the invention, the vector is an adenovirus vector. An adenovirus according to the invention belongs to the family of the Adenoviridae, and preferably is one that belongs to the genus Mastadenovirus. It can be a human adenovirus, but also an adenovirus that infects other species, including but not limited to a bovine adenovirus (e.g., bovine adenovirus 3, BAdV3), a canine adenovirus (e.g., CAdV2), a porcine adenovirus (e.g., PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g., the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials.

Simian adenoviruses generally also have a low seroprevalence and/or low pre-existing neutralizing antibody titers in the human population, and a significant amount of work has been reported using chimpanzee adenovirus vectors (e.g., U.S. Pat. No. 6,083,716; WO 2005/071093; WO 2010/086189; WO 2010/085984; Farina et al, 2001, J Virol 75:11603-13; Cohen et al, 2002, J Gen Virol 83:151-55; Kobinger et al, 2006, Virology 346:394-401; Tatsis et al., 2007, Molecular Therapy 15:608-17; see also review by Bangari and Mittal, 2006, Vaccine 24:849-62; and review by Lasaro and Ertl, 2009, Mol Ther 17:1333-39). Hence, in other embodiments, the recombinant adenovirus according to the invention is based upon a simian adenovirus, e.g. a chimpanzee adenovirus. In certain embodiments, the recombinant adenovirus is based upon simian adenovirus type 1, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P. In certain embodiments, the recombinant adenovirus is based upon a chimpanzee adenovirus such as ChAdOx 1 (see, e.g., WO 2012/172277), or ChAdOx 2 (see, e.g., WO 2018/215766). In certain embodiments, the recombinant adenovirus is based upon a chimpanzee adenovirus such as BZ28 (see, e.g., WO 2019/086466). In certain embodiments, the recombinant adenovirus is based upon a gorilla adenovirus such as BLY6 (see, e.g., WO 2019/086456), or BZ1 (see, e.g., WO 2019/086466).

In a preferred embodiment of the invention, the adenoviral vectors comprise capsid proteins from rare serotypes, e.g. including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton, and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g. WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively)

In certain preferred embodiments the recombinant adenovirus vector useful in the invention is derived mainly or entirely from Ad26 (i.e., the vector is rAd26). In some embodiments, the adenovirus is replication deficient, e.g., because it contains a deletion in the E1 region of the genome. For adenoviruses being derived from non-group C adenovirus, such as Ad26 or Ad35, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g., Havenga, et al., 2006, J Gen Virol 87:2135-43; WO 03/104467). However, such adenoviruses will not be capable of replicating in non-complementing cells that do not express the E1 genes of Ad5.

The preparation of recombinant adenoviral vectors is well known in the art. Preparation of rAd26 vectors is described, for example, in WO 2007/104792 and in Abbink et al., (2007) Virol 81 (9): 4654-63. Exemplary genome sequences of Ad26 are found in GenBank Accession EF 153474 and in SEQ ID NO: 1 of WO 2007/104792. Examples of vectors useful for the invention for instance include those described in WO2012/082918, the disclosure of which is incorporated herein by reference in its entirety.

Typically, a vector useful in the invention is produced using a nucleic acid comprising the entire recombinant adenoviral genome (e.g., a plasmid, cosmid, or baculovirus vector). Thus, the invention also provides isolated nucleic acid molecules that encode the adenoviral vectors of the invention. The nucleic acid molecules of the invention can be in the form of RNA or in the form of DNA obtained by cloning or produced synthetically. The DNA can be double-stranded or single-stranded.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding the stabilized pre-fusion PIV1 F protein (usually linked to a promoter), or a gene encoding the pre-fusion PIV1 F protein fragment (usually linked to a promoter) within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines for adenoviruses with a deletion in the E1 region include, for example, PER. C6, 911, 293, and E1 A549.

In a preferred embodiment of the invention, the vector is an adenovirus vector, and more preferably a rAd26 vector, most preferably a rAd26 vector with at least a deletion in the E1 region of the adenoviral genome, e.g. such as that described in Abbink, J Virol, 2007. 81 (9): p. 4654-63, which is incorporated herein by reference. Typically, the nucleic acid sequence encoding the pre-fusion PIV1 F protein is cloned into the E1 and/or the E3 region of the adenoviral genome.

Host cells comprising the nucleic acid molecules encoding the pre-fusion PIV1 F proteins form also part of the invention. The pre-fusion PIV1 F proteins may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells. In certain embodiments, the cells are human cells. In general, the production of a recombinant proteins, such the pre-fusion PIV1 F proteins of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here the pre-fusion PIV1 F proteins. The suitable medium may or may not contain serum.

A “heterologous nucleic acid molecule” (also referred to herein as ‘transgene’) is a nucleic acid molecule that is not naturally present in the host cell. It is introduced into for instance a vector by standard molecular biology techniques. A transgene is generally operably linked to expression control sequences. This can for instance be done by placing the nucleic acid encoding the transgene(s) under the control of a promoter. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, polyA sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R.I. Freshney, Culture of animal cells: A manual of basic technique, fourth edition (Wiley-Liss Inc., 2000, ISBN 0-471-34889-9)).

The invention further provides compositions comprising a pre-fusion PIV1 F protein, and/or fragment thereof, and/or a nucleic acid molecule, and/or a vector, as described herein. The invention thus provides compositions comprising a pre-fusion PIV1 F protein, or fragment thereof, that displays an epitope that is present in a pre-fusion conformation of the PIV1 F protein but is absent in the post-fusion conformation. The invention also provides compositions comprising a nucleic acid molecule and/or a vector, encoding such pre-fusion PIV1 F protein or fragment. The invention further provides pharmaceutical compositions, e.g. vaccine compositions, comprising a pre-fusion PIV1 F protein, a PIV1 F protein fragment, and/or a nucleic acid molecule, and/or a vector, as described above and one or more pharmaceutically acceptable excipients.

The invention also provides the use of a stabilized pre-fusion PIV1 F protein (fragment), a nucleic acid molecule, and/or a vector, according to the invention, for inducing an immune response against PIV1 F protein in a subject. Further provided are methods for inducing an immune response against PIV1 F protein in a subject, comprising administering to the subject a pre-fusion PIV1 F protein (fragment), and/or a nucleic acid molecule, and/or a vector, according to the invention. Also provided are pre-fusion PIV1 F protein (fragments), nucleic acid molecules, and/or vectors, according to the invention for use in inducing an immune response against PIV1 F protein in a subject. Further provided is the use of the pre-fusion PIV1 F protein (fragments), and/or nucleic acid molecules, and/or vectors according to the invention for the manufacture of a medicament for use in inducing an immune response against PIV1 F protein in a subject. The invention in particular provides pre-fusion PIV1 F protein (fragments), and/or nucleic acid molecules, and/or vectors according to the invention for use as a vaccine.

The pre-fusion PIV1 F protein (fragments), nucleic acid molecules, or vectors of the invention may be used for prevention (prophylaxis) and/or treatment of PIV1 infections. In certain embodiments, the prevention and/or treatment may be targeted at patient groups that are susceptible PIV1 infection. Such patient groups include, but are not limited to e.g., the elderly (e.g. ≥50 years old, ≥60 years old, and preferably ≥65 years old), the young (e.g. ≤5 years old, ≤1 year old), pregnant women (for maternal immunization), and hospitalized patients and patients who have been treated with an antiviral compound but have shown an inadequate antiviral response.

The pre-fusion PIV1 F proteins, fragments, nucleic acid molecules and/or vectors according to the invention may be used in stand-alone treatment and/or prophylaxis of a disease or condition caused by PIV1, or in combination with other prophylactic and/or therapeutic treatments, such as (existing or future) vaccines, antiviral agents and/or monoclonal antibodies.

The invention further provides methods for preventing and/or treating PIV1 infection in a subject utilizing the pre-fusion PIV1 F proteins or fragments thereof, nucleic acid molecules and/or vectors according to the invention. In a specific embodiment, a method for preventing and/or treating PIV1 infection in a subject comprises administering to a subject in need thereof an effective amount of a pre-fusion PIV1 F protein (fragment), nucleic acid molecule and/or a vector, as described above. A therapeutically effective amount refers to an amount of a protein, nucleic acid molecule or vector, that is effective for preventing, ameliorating and/or treating a disease or condition resulting from infection by PIV1. Prevention encompasses inhibiting or reducing the spread of PIV1 or inhibiting or reducing the onset, development or progression of one or more of the symptoms associated with infection by PIV1. Amelioration as used in herein may refer to the reduction of visible or perceptible disease symptoms, viremia, or any other measurable manifestation of PIV1 infection.

For administering to subjects, such as humans, the invention may employ pharmaceutical compositions comprising a pre-fusion PIV1 F protein (fragment), a nucleic acid molecule and/or a vector as described herein, and a pharmaceutically acceptable carrier or excipient. In the present context, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The PIV1 F proteins, or nucleic acid molecules, preferably are formulated and administered as a sterile solution although it may also be possible to utilize lyophilized preparations. Sterile solutions are prepared by sterile filtration or by other methods known per se in the art. The solutions are then lyophilized or filled into pharmaceutical dosage containers. The pH of the solution generally is in the range of pH 3.0 to 9.5, e.g. pH 5.0 to 7.5. The PIV1 F proteins typically are in a solution having a suitable pharmaceutically acceptable buffer, and the composition may also contain a salt. Optionally stabilizing agent may be present, such as albumin. In certain embodiments, detergent is added. In certain embodiments, the PIV1 F proteins may be formulated into an injectable preparation.

In certain embodiments, a composition according to the invention further comprises one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance an immune response to the PIV1 F proteins of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc), which stimulate immune response upon interaction with recipient cells. In certain embodiments the compositions of the invention comprise aluminium as an adjuvant, e.g. in the form of aluminium hydroxide, aluminium phosphate, aluminium potassium phosphate, or combinations thereof, in concentrations of 0.05-5 mg, e.g. from 0.075-1.0 mg, of aluminium content per dose.

In other embodiments, the compositions do not comprise adjuvants.

In certain embodiments, the invention provides methods for making a vaccine against respiratory syncytial virus (PIV1), comprising providing an PIV1 F protein (fragment), nucleic acid or vector according to the invention and formulating it into a pharmaceutically acceptable composition. The term “vaccine” refers to an agent or composition containing an active component effective to induce a certain degree of immunity in a subject against a certain pathogen or disease, which will result in at least a decrease (up to complete absence) of the severity, duration or other manifestation of symptoms associated with infection by the pathogen or the disease. In the present invention, the vaccine comprises an effective amount of a pre-fusion PIV1 F protein (fragment) and/or a nucleic acid molecule encoding a pre-fusion PIV1 F protein, and/or a vector comprising said nucleic acid molecule, which results in an effective immune response against PIV1. This provides a method of preventing serious lower respiratory tract disease leading to hospitalization and the decrease in frequency of complications such as pneumonia and bronchiolitis due to PIV1 infection and replication in a subject. The term “vaccine” according to the invention implies that it is a pharmaceutical composition, and thus typically includes a pharmaceutically acceptable diluent, carrier or excipient. It may or may not comprise further active ingredients. In certain embodiments it may be a combination vaccine that further comprises other components that induce an immune response, e.g. against other proteins of PIV1 and/or against other infectious agents, e.g. against RSV, HMPV and/or influenza. The administration of further active components may for instance be done by separate administration or by administering combination products of the vaccines of the invention and the further active components.

A subject as used herein preferably is a mammal, for instance a rodent, e.g. a mouse, a cotton rat, or a non-human-primate, or a human. Preferably, the subject is a human subject.

The invention further provides methods for making a vaccine against PIV1, comprising providing a recombinant human adenovirus of serotype 26 that comprises nucleic acid encoding a pre-fusion PIV1 F protein or fragment thereof as described herein, propagating said recombinant adenovirus in a culture of host cells, isolating and purifying the recombinant adenovirus, and bringing the recombinant adenovirus in a pharmaceutically acceptable composition. In certain embodiments, provided herein are methods of producing an adenoviral particle comprising a nucleic acid molecule encoding a PIV1 F protein or fragment thereof (transgene). The methods comprise (a) contacting a host cell of the invention with an adenoviral vector of the invention and (b) growing the host cell under conditions wherein the adenoviral particle comprising the transgene is produced. Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

Most large-scale suspension cultures are operated as batch or fed-batch processes because they are the most straightforward to operate and scale up. Nowadays, continuous processes based on perfusion principles are becoming more common and are also suitable (see, e.g., WO 2010/060719, and WO 2011/098592, both incorporated by reference herein, which describe suitable methods for obtaining and purifying large amounts of recombinant adenoviruses).

The invention further provides an isolated recombinant nucleic acid that forms the genome of a recombinant human adenovirus of serotype 26 that comprises nucleic acid encoding a PIV1 F protein or fragment thereof, as described herein.

In addition, the proteins of the invention may be used as diagnostic tool, for example to test the immune status of an individual by establishing whether there are antibodies in the serum of such individual capable of binding to the protein of the invention. The invention thus also relates to an in vitro diagnostic method for detecting the presence of an PIV1 infection in a patient said method comprising the steps of a) contacting a biological sample obtained from said patient with a protein according to the invention; and b) detecting the presence of antibody-protein complexes.

The invention is further illustrated in the following examples. The examples do not limit the invention in any way. They merely serve to clarify the invention.

### EXAMPLES

### Example 1. Stabilizing Mutations Allow Trimeric Expression of Soluble HPIV1 F Ectodomain without a Heterologous Trimerization Domain, as Analyzed with Analytical SEC

To stabilize the HR2 region of HPIV1 F and allow HPIV1 F ectodomain expression in the absence of a trimerization domain, amino acid residues at position 473 and 480 in the HR2 stem region (comprising amino acid residues 456-488) were mutated into hydrophobic amino acids, in particular into V (S473V+A480V). In the HR2-stabilized backbone, the impact of additional amino acid substitutions at positions 38, 40, 44, 134, 168, 169, 170, 171, 172, 175, 218, 228, and 323, i.e. in the so-called head region (comprising amino acid residues 22-455), and positions 469, 478, 483, in the HR2 stem region, was assessed.

Plasmids encoding HPIV1 F protein ectodomain in which the transmembrane and cytoplasmic tail were replaced with a C-tag (SEQ ID NO: 2) were synthesized and codon-optimized at Genscript. The constructs were cloned into pCDNA2004 by standard methods widely known within the field involving site-directed mutagenesis and PCR and sequenced. Proteins were expressed in the Expi293F cell system. Expi293F cells were transiently transfected using ExpiFectamine (Life Technologies) according to the manufacturer's instructions and cultured for 3 days at 37° C. and 10% CO₂. The culture supernatant was collected, and cells and cellular debris were removed by centrifugation for 5 minutes at 300 g. The clarified supernatant was subsequently sterile filtered using a 0.22 μm filter. The cell culture supernatants of the different HPIV1 F variants were analyzed using analytical size exclusion chromatography (SEC). An ultra high-performance liquid chromatography system (Vanquish, Thermo Scientific) and μDAWN TREOS instrument (Wyatt) coupled to an Optilab μT-rEX Refractive Index Detector (Wyatt) in combination with an in-line Nanostar DLS reader (Wyatt) was used for performing the analytical SEC experiment. The cleared crude cell culture supernatants were applied to a 300 Å column, (Sepax) with the corresponding guard column (Sepax) equilibrated in running buffer (150 mM sodium phosphate, 50 mM NaCl, pH 7.0) at 0.35 mL/min. When analyzing supernatant samples, μMALS detectors were offline and analytical SEC data was analyzed using Chromeleon 7.2.8.0 software package.

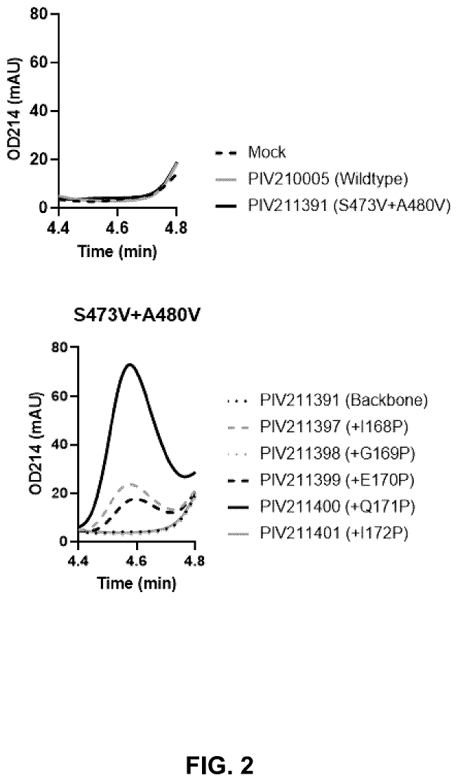

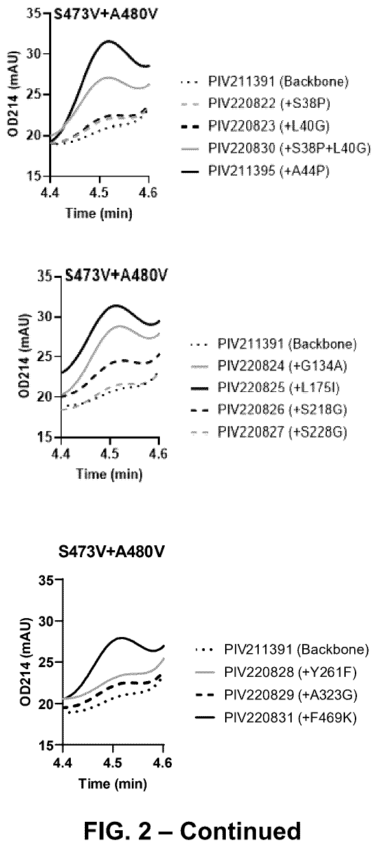

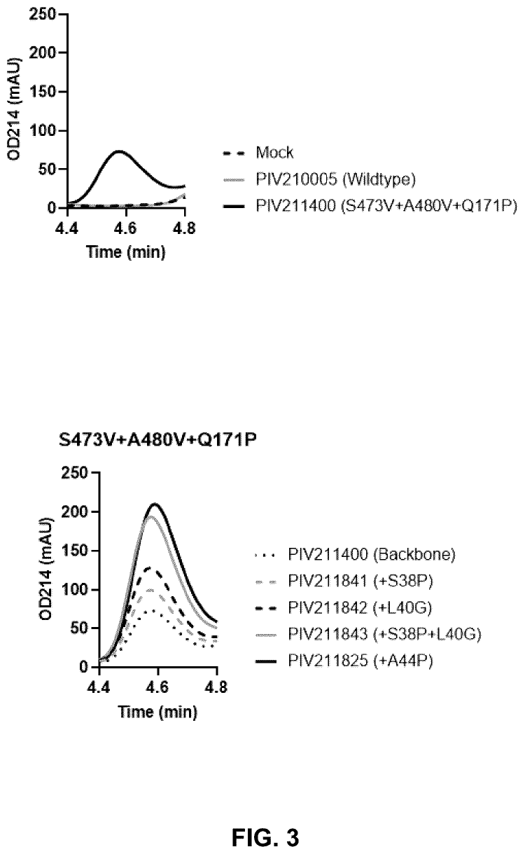

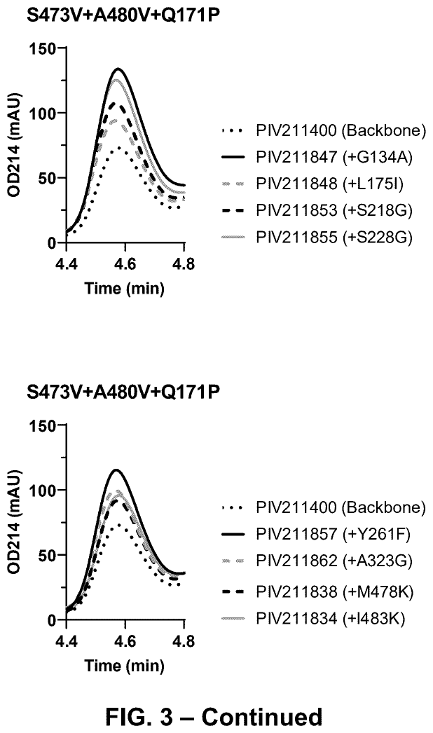

HPIV1 F trimer was not detected upon expression of neither wild type ectodomain F (PIV210005) nor of HR2 stabilized variant PIV211391 (FIG. 2A,2B, top panels). When single proline substitutions were systematically added to region 168-172 in the head domain (comprising amino acid residues 22-455) of HPIV1 F, trimer could be detected for 1168P, E170P and in particular for Q171P mutations, but not for G169P and 1172P substitutions (FIG. 2A, bottom panel). Amino acid substitutions A44P, G134A, L175I, and S218G in the head region of HPIV1 F, and F469K in the HR2 region of HPIV1 F also yielded detectable HPIV1 F trimer compared to PIV211391 (FIG. 2B). In contrast, a positive impact of amino acid substitutions S38P, L40G, S228G, Y261F, and A323G on HPIV1 F trimer yield could only be detected when a more stabilized backbone including Q171P+S473V+A480V was employed (PIV211400) (compare FIG. 2B to FIG. 3), or when S38P+L40G were combined in backbone PIV211391 (FIG. 2B). Introduction of M478K and I483 stabilizing mutations in the HR2 region of the PIV211400 backbone was also shown to increase trimer expression (FIG. 3).

### Example 2. Increased HPIV1 F Trimer Expression and Stability by the Combination of Amino Acid Substitutions, as Analyzed with Analytical SEC and DSF

Trimeric HPIV1 F ectodomain expression in the absence of a trimerization domain was detected upon introduction of HR2 mutations S473V+A480V and either head domain mutations Q171P+G38P+L40G (PIV211843) or Q171P+G134A (PIV211847). These two backbones were employed to assess the effect of additional amino acid substitutions at positions 38+40, 134, 175, 218, 228, 261, or 323 on HPIV1 F trimer expression and stability. To this end, plasmids coding for recombinant HPIV1 F protein ectodomains equipped with a C-tag were expressed in Expi293F cells, and 3 days after transfection the supernatants were analyzed for trimer content using analytical SEC, as described in example 1. Melting temperature (Tm50) as a measure of protein stability was determined by Differential Scanning Fluorimetry (DSF). To this end, the fluorescent emission of Sypro Orange Dye (Thermo Fisher Scientific) added to HPIV1 F protein in crude supernatant was monitored. The measurement was performed with a starting temperature of 25° C. and a final temperature of 95° C. (54° C. increase per hour). Melting curves were measured using a ViiA7 real-time PCR machine (Applied Biosystems), and Tm50 values were derived from the negative first derivative as described previously (Rutten et al. (2020) Cell Rep, 30:4540-4550).

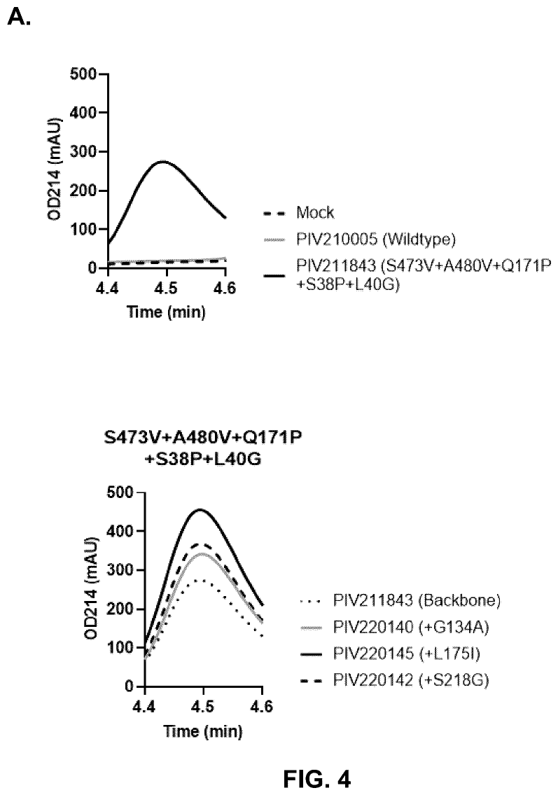

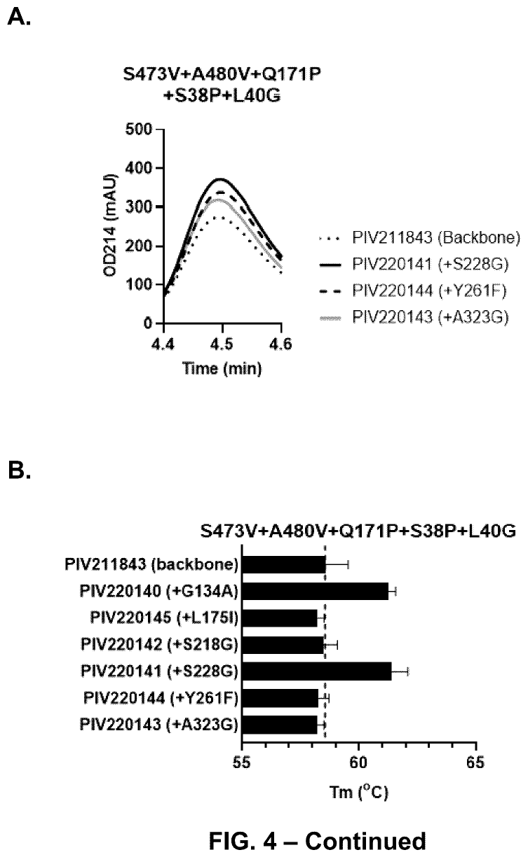

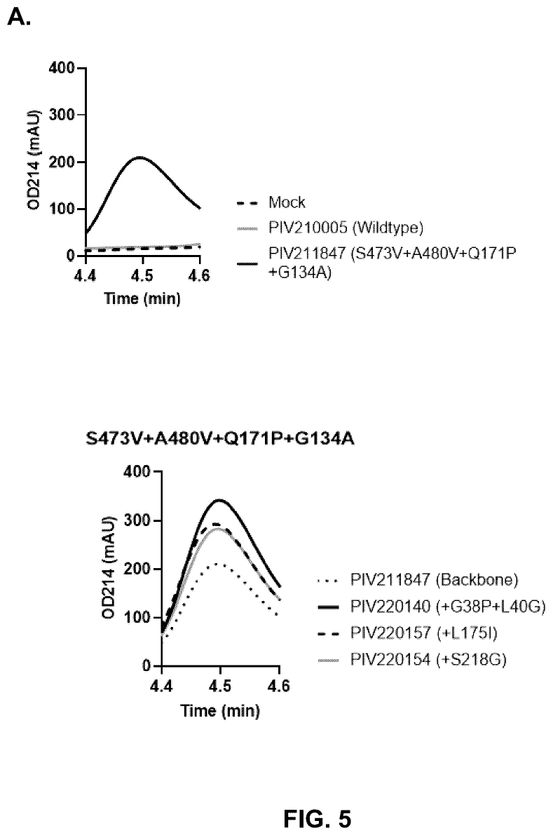

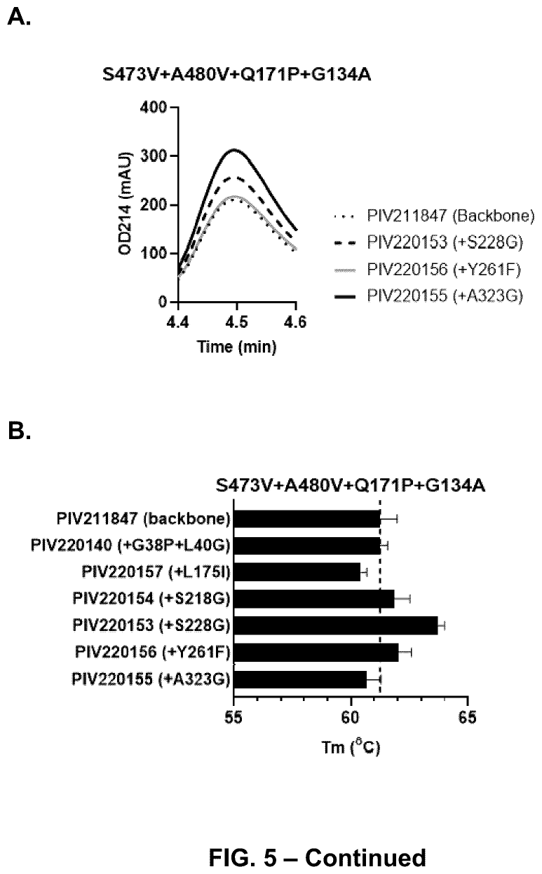

The additive effect of individual mutations G134A, L175I, S218G, S228G, Y261F, and A323G on HPIV1 F trimer expression was demonstrated in backbone PIV211843, carrying S473V+A480V+Q171P+G38P+L40G (FIG. 4A). Similarly, the additive effect of mutations G38P+L40G, L175I, S218G, S228G, and A323G on HPIV1 F trimer expression was demonstrated in backbone PIV211847, carrying S473V+A480V+Q171P+G134A, while no positive impact of Y261F on trimer expression was shown for this particular backbone (FIG. 5A). The stability of HPIV1 F trimer in supernatant was assessed by DSF and demonstrated a 2.7- and 2.8-degree Celsius increase in melting temperature upon introduction of G134A or S228G, respectively (FIG. 4B), while other substitutions did not impact Tm50 (FIG. 4B). When G134A and S228G were combined in PIV220153, the HPIV1 F melting temperature increased further by 2.5° C., demonstrating the additive effect of these two mutations on trimer stability (FIG. 5B).

### Example 3. Purification and Characterization of Stabilized Pre-Fusion HPIV1 F Trimer without a Trimerization Domain

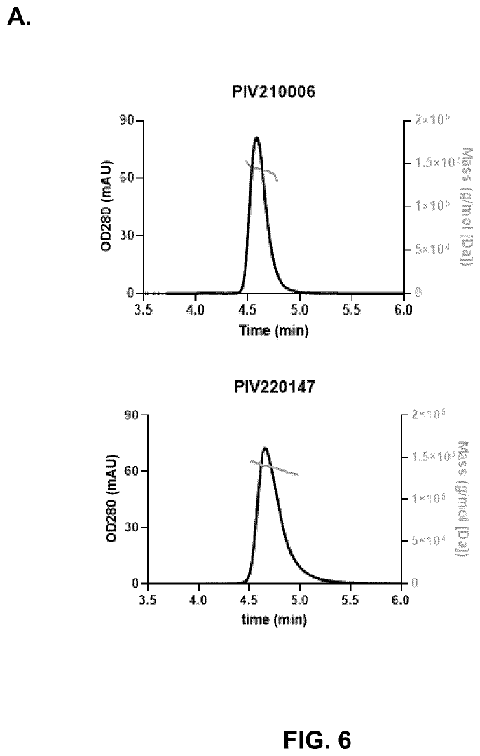

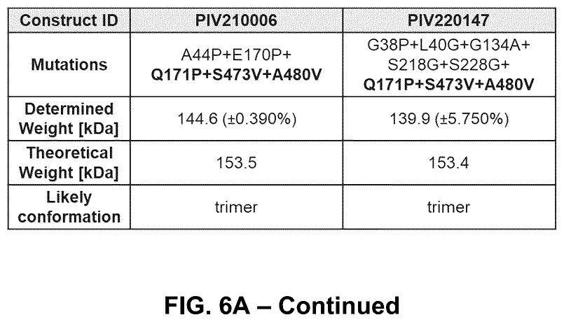

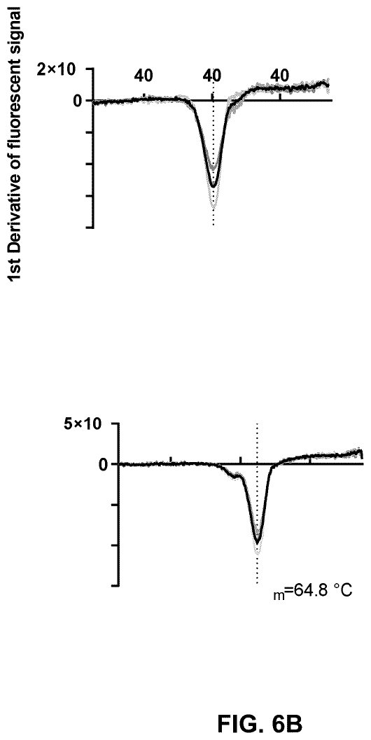

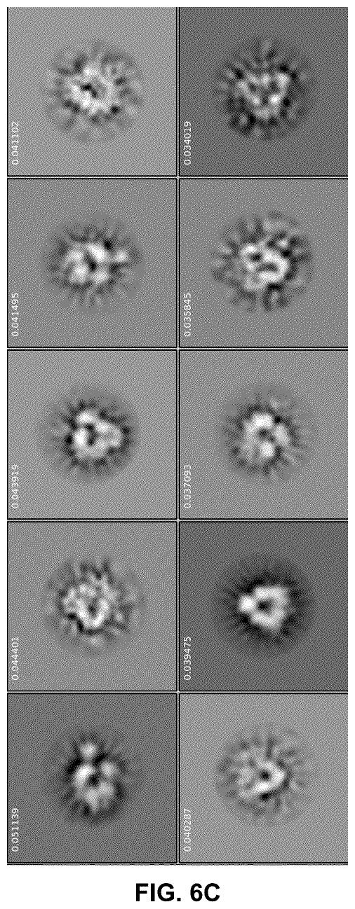

Two HPIV1 F ectodomain variants without a heterologous trimerization domain and with varying stabilizing mutations were selected for purification and further characterization. To this end, PIV210006, carrying A44P+E170P+Q171P+S473V+A480V, and PIV220147, carrying G38P+L40G+G134A+Q171P+S218G+S228G+S473V+A480V, were transiently expressed in Expi293 cells using ExpiFectamine (Life Technologies) according to the manufacturer's instructions and culturing for 5 days at 37° C. and 10% CO₂. HPIV1 F trimer was purified from sterile-filtered crude cell culture supernatant using a two-step purification protocol including CaptureSelect™ C-tagXL affinity column, followed by size-exclusion chromatography using a Superdex200 10/300 column (Cytiva). The trimeric fraction was pooled and further characterized by SEC-MALS using an ultra-high-performance liquid chromatography system (Vanquish, Thermo Scientific) and μDAWN TREOS instrument (Wyatt) coupled to an Optilab μT-rEX Refractive Index Detector (Wyatt), in combination with an in-line Nanostar DLS reader (Wyatt). Protein was loaded onto a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package, and conformation and molecular weight of HPIV1 F trimers was calculated by Astra software and compared to the calculated weight. Tm50 of purified HPIV1 F trimer was determined by DSF as described in example 2. Characterization of the pooled fractions demonstrated a single trimeric peak with expected molecular weight of approximately 150 kDa (FIG. 6A). PIV220147 had a higher Tm50 than PIV210006; respectively 64.9° C. and 60.4° C., which is in line with the presence of temperature stability-increasing substitutions G134A and S228G, in PIV220147 (FIG. 6B).

The conformation of the F trimer of PIV220147 was further examined by negative stain transmission electron microscopy (nsTEM), followed by two-dimensional (2D) class averaging of acquired images. PIV220147 was diluted to a concentration of 20 μg/mL in 20 mM Tris and 150 mM sodium chloride, pH 7.4, and a 4-μL sample was adhered onto a carbon-coated 200-mesh copper grid (Electron Microscopy Sciences) that had been glow discharged (Pelco easiGlow, 25 mA for 45 s) prior to use. The sample drop was applied for 1 min and subsequently blotted with a filter paper (Whatman no. 1 or 4). Grids were stained with 3 μL 2% uranyl acetate in filtered Milli-Q (filtered with Millipore filter 0.22 μm) for 60 s and blotted with filter paper (Whatman no. 1 or 4). Data were collected using a Talos L120C electron microscope operating at 120 keV with a magnification of 73,000x. Images were acquired on a Ceta camera (Thermo Fisher Scientific). For 2D class-averaged images, 2839 images were collected, and 1062220 particles were picked, classified, and averaged using RELION4.0-beta. 2D averaging of nsTEM images showed that the protein had a globular shape that mimics the typical paramyxovirus pre-fusion F structure. No typical cone-shaped proteins were detected that would have corresponded to the post-fusion structure.

TABLE 1
Standard amino acids, abbreviations and properties

| | | | Side chai [symbol] | Side chain | Hydrophobic/ |
|---|---|---|---|---|---|
| Amino Acid | 3-Lette [symbol] | 1-Lette [symbol] | polarity | charge (pH 7.4) | hydrophilic |
| alanine | Ala | A | non-polar | Neutral | hydrophobic |
| arginine | Arg | R | polar | Positive | hydrophilic |
| asparagine | Asn | N | polar | Neutral | hydrophilic |
| aspartic acid | Asp | D | polar | Negative | hydrophilic |
| cysteine | Cys | C | non-polar | Neutral | hydrophobic |
| glutamic acid | Glu | E | polar | Negative | hydrophilic |
| glutamine | Gln | Q | polar | Neutral | hydrophilic |
| glycine | Gly | G | non-polar | Neutral | hydrophobic |
| histidine | His | H | polar | positive(10%) | hydrophilic |
| | | | | neutral(90%) | |
| isoleucine | Ile | I | non-polar | Neutral | hydrophobic |
| leucine | Leu | L | non-polar | Neutral | hydrophobic |
| lysine | Lys | K | polar | Positive | hydrophilic |
| methionine | Met | M | non-polar | Neutral | hydrophobic |
| phenylalanine | Phe | F | non-polar | Neutral | hydrophobic |
| proline | Pro | P | non-polar | Neutral | hydrophobic |
| serine | Ser | S | polar | Neutral | hydrophilic |
| threonine | Thr | T | polar | Neutral | hydrophilic |
| tryptophan | Trp | W | non-polar | Neutral | hydrophobic |
| tyrosine | Tyr | Y | polar | Neutral | hydrophobic |
| valine | Val | V | non-polar | Neutral | hydrophobic |

[symbol]

indicates data missing or illegible when filed

SEQUENCES

SEQ ID NO: 1 - Full-length (membrane-bound) wildtype PIV1 F protein
MQSSEILLLVYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEESKTELM
KARAIISAVGGWHNKESTQIIIIIIVCILIIIICGILYYLYRVRRLLIMINSTNNSPINAYTLE
SRMKNPYMGNHSNKIRSSTLHTYNNQIYPQLLSDVVRK
SEQ ID NO: 2 - Ectodomain without the signal peptide and truncated after residue 488
QIPVDKLSNVGVIINEGKLLKIAGSYESRYIVLSLVPSIDLQDGCGTTQIIQYKNLLNRL
LIPLKDALDLQESLITITNDTTVTNDNPQTRFFGAVIGTIALGVATAAQITAGIALAEA
REARKDIALIKDSIVKTHNSVEFIQRGIGEQIIALKTLQDFVNDEIRPAIGELRCETTAL
KLGIKLTQHYSELATAFSSNLGTIGEKSLTLQALSSLYSANITEILSTIKKDKSDIYDIIY
TEQVKGTVIDVDLEKYMVTLLVKIPILSEIPGVLIYRASSISYNIEGEEWHVAIPNYIISK
ASSLGGADVTNCIESKLAYICPRDPTQLIPDNQQKCILGDVSKCPVTKVINNLVPKFAF
INGGVVANCIASTCTCGTNRIPVNQDRSKGVTFLTYTNCGLIGINGIELYANKRGRDT
TWGNQIIKVGPAVSIRPVDISLNLASATNFLEESKTELMKARAIISAVG
SEQ ID NO: 3 - PIV210005: PIV1 F_Wildtype_C-tag
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEESKTELM
KARAIISAVGSGEPEA
SEQ ID NO: 4 - PIV210006
MQSSEILILAYSSLLLSSSLCQIPVDKLSNVGVIINEGKLLKIPGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGPPIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEVKTELMKV
RAIISAVGSGEPEA
SEQ ID NO: 5 - PIV211391
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 6 - PIV220831
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNKLEEVKTEL
MKVRAIISAVGSGEPEA
SEQ ID NO: 7 - PIV211395
MQSSEILILAYSSLLLSSSLCQIPVDKLSNVGVIINEGKLLKIPGSYESRYIVLSLVPSIDL
QDGCGTTQIIQYKNLLNRLLIPLKDALDLQESLITITNDTTVTNDNPQTRFFGAVIGTIA
LGVATAAQITAGIALAEAREARKDIALIKDSIVKTHNSVEFIQRGIGEQIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEVKTELMKV
RAIISAVGSGEPEA
SEQ ID NO: 8 - PIV211397
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGPGEQIIALKTLQD
FVNDEIRPAIGELRCETTALKLGIKLTQHYSELATAFSSNLGTIGEKSLTLQALSSLYS
ANITEILSTIKKDKSDIYDIIYTEQVKGTVIDVDLEKYMVTLLVKIPILSEIPGVLIYRAS
SISYNIEGEEWHVAIPNYIISKASSLGGADVTNCIESKLAYICPRDPTQLIPDNQQKCIL
GDVSKCPVTKVINNLVPKFAFINGGVVANCIASTCTCGTNRIPVNQDRSKGVTFLTYT
NCGLIGINGIELYANKRGRDTTWGNQIIKVGPAVSIRPVDISLNLASATNFLEEVKTEL
MKVRAIISAVGSGEPEA
SEQ ID NO: 9 - PIV211398
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIPEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 10 - PIV211399
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGPQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 11 - PIV211400
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 12 - PIV211401
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQPIALKTLQD
FVNDEIRPAIGELRCETTALKLGIKLTQHYSELATAFSSNLGTIGEKSLTLQALSSLYS
ANITEILSTIKKDKSDIYDIIYTEQVKGTVIDVDLEKYMVTLLVKIPILSEIPGVLIYRAS
SISYNIEGEEWHVAIPNYIISKASSLGGADVTNCIESKLAYICPRDPTQLIPDNQQKCIL
GDVSKCPVTKVINNLVPKFAFINGGVVANCIASTCTCGTNRIPVNQDRSKGVTFLTYT
NCGLIGINGIELYANKRGRDTTWGNQIIKVGPAVSIRPVDISLNLASATNFLEEVKTEL
MKVRAIISAVGSGEPEA
SEQ ID NO: 13 - PIV211825
MQSSEILILAYSSLLLSSSLCQIPVDKLSNVGVIINEGKLLKIPGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGEPIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEVKTELMKV
RAIISAVGSGEPEA
SEQ ID NO: 14 - PIV211834
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVINCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAKISAVGSGEPEA
SEQ ID NO: 15 - PIV211838
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELK
KVRAIISAVGSGEPEA
SEQ ID NO: 16 - PIV211841
MQSSEILILAYSSLLLSSSLCQIPVDKLSNVGVIINEPKLLKIAGSYESRYIVLSLVPSIDL
QDGCGTTQIIQYKNLLNRLLIPLKDALDLQESLITITNDTTVTNDNPQTRFFGAVIGTIA
LGVATAAQITAGIALAEAREARKDIALIKDSIVKTHNSVEFIQRGIGEPIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEVKTELMKV
RAIISAVGSGEPEA
SEQ ID NO: 17 - PIV211842
MQSSEILILAYSSLLLSSSLCQIPVDKLSNVGVIINEGKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 18 - PIV211843
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 19 - PIV211847
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 20 - PIV211848
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEPIIAIKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 21 - PIV211853
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFGSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 22 - PIV211855
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKGLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 23 - PIV211857
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIFTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 24 - PIV211862
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKGSSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 25 - PIV220140
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVINDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 26 - PIV220141
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKGLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 27 - PIV220142
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFGSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 28 - PIV220143
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKGSSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 29 - PIV220144
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIFTEQVKGTVIDVDLEKYMVTLLVKIPILSEIPGVLIYRASSI
SYNIEGEEWHVAIPNYIISKASSLGGADVINCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 30 - PIV220145
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIAIKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 31 - PIV220147
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFGSNLGTIGEKGLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 32 - PIV220153
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFSSNLGTIGEKGLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 33 - PIV220154
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFGSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 34 - PIV220155
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKGSSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 35 - PIV220156
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAAIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIFTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 36 - PIV220157
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAAIALAEAREARKDIALIKDSIVKTHNSVEFIQRGIGEPIIAIKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 37 - PIV220822
MQSSEILILAYSSLLLSSSLCQIPVDKLSNVGVIINEPKLLKIAGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGEQIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEVKTELMKV
RAIISAVGSGEPEA
SEQ ID NO: 38 - PIV220823
MQSSEILILAYSSLLLSSSLCQIPVDKLSNVGVIINEGKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 39 - PIV220824
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAAIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 40 - PIV220825
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIAIKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 41 - PIV220826
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVINDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFGSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 42 - PIV220827
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKGLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 43 - PIV220828
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIFTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 44 - PIV220829
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKGSSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 45 PIV220830
MQSSEILILAYSSLLLSSSLCQIPVDKLSNVGVIINEPKGLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEVKTELM
KVRAIISAVGSGEPEA
SEQ ID NO: 46 - PIV220831
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNKLEEVKTEL
MKVRAIISAVGSGEPEA
SEQ ID NO: 47 - PIV220150
MQSSEILILAYSSLLLSSSLCQIPVDKLSNVGVIINEpKgLKIAGSYESRYIVLSLVPSIDL
QDGCGTTQIIQYKNLLNRLLIPLKDALDLQESLITITNDTTVINDNPQTRFFGAVIGTIA
LGVATAAQITAaIALAEAREARKDIALIKDSIVKTHNSVEFIQRGIGEpIIALKTLQDFV
NDEIRPAIGELRCETTALKLGIKLTQHYSELATAFgSNLGTIGEKgLTLQALSSLYSANI
TEILSTIKKDKSDIYDIIYTEQVKGTVIDVDLEKYMVTLLVKIPILSEIPGVLIYRASSISY
NIEGEEWHVAIPNYIISKgSSLGGADVINCIESKLAYICPRDPTQLIPDNQQKCILGDVS
KCPVTKVINNLVPKFAFINGGVVANCIASTCTCGTNRIPVNQDRSKGVTFLTYTNCGL
IGINGIELYANKRGRDTTWGNQIIKVGPAVSIRPVDISLNLASATNFLEEvKTELMKvR
AIISAVGSGEPEA
SEQ ID NO: 48 - PIV220151
MQSSEILILAYSSLLLSSSLCQIPVDKLSNVGVIINEpKgLKIAGSYESRYIVLSLVPSIDL
QDGCGTTQIIQYKNLLNRLLIPLKDALDLQESLITITNDTTVTNDNPQTRFFGAVIGTIA
LGVATAAQITAaIALAEAREARKDIALIKDSIVKTHNSVEFIQRGIGEpIIAiKTLQDFVN
DEIRPAIGELRCETTALKLGIKLTQHYSELATAFgSNLGTIGEKgLTLQALSSLYSANIT
EILSTIKKDKSDIYDIIfTEQVKGTVIDVDLEKYMVTLLVKIPILSEIPGVLIYRASSISYN
IEGEEWHVAIPNYIISKgSSLGGADVTNCIESKLAYICPRDPTQLIPDNQQKCILGDVSK
CPVTKVINNLVPKFAFINGGVVANCIASTCTCGTNRIPVNQDRSKGVTFLTYTNCGLI
GINGIELYANKRGRDTTWGNQIIKVGPAVSIRPVDISLNLASATNFLEEvKTELMKvRA
IISAVGSGEPEA

1. Human parainfluenza virus 1 (HPIV1) F protein, comprising an F1 and an F2 domain, or a fragment thereof, comprising an amino acid sequence of the F1 and F2 domain of an F protein of an HPIV1 strain, comprising an hydrophobic amino acid at position 473 and at position 480, wherein said hydrophobic amino acid is selected from the group consisting of valine (V), isoleucine (I), leucine (L) and methionine (M), and wherein the amino acid residue at position 171 is P, the amino acid residue at position 44 is P, the amino acid residue at position 134 is A, the amino acid residue at position 175 is I, the amino acid residue at position 218 is G, the amino acid residue at position 469 is K, the amino acid residue at position 168 is P, the amino acid residue at position 170 is P, the amino acid residue at position 38 P, and/or the amino acid residue at position 40 is G, and/or the amino acid at position 38 is P and the amino acid residue at position 40 is G, wherein the numbering of the amino acid positions is according to the numbering of the amino acid residues in SEQ ID NO: 1.

2. Protein or fragment according to claim 1, comprising a hydrophobic amino acid at position 473 and at position 480, wherein said hydrophobic amino acid is selected from the group consisting of valine (V), isoleucine (I), leucine (L) and methionine (M), and wherein the amino acid residue at position 171 is P.

3. Protein or fragment according to claim 2, wherein the amino acid residue at position 38 is P, the amino acid at position 40 is G, the amino acid residue at position 44 is P, the amino acid residue at position 134 is A, the amino acid residue at position 175 is I, the amino acid at position 218 is G, the amino acid residue at position 228 is G, the amino acid residue at position 261 is F, the amino acid residue at position 478 is K, the amino acid residue at position 483 is K and/or the amino acid residue at position 323 is G, and/or the amino acid at position 38 is P and the amino acid residue at position 40 is G.

4. Protein or fragment according to claim 2, comprising a hydrophobic amino acid at position 473 and at position 480, wherein said hydrophobic amino acid is selected from the group consisting of valine (V), isoleucine (I), leucine (L) and methionine (M), and wherein the amino acid residue at position 171 is P and the amino acid at position 38 is P and the amino acid at position 40 is G.

5. Protein or fragment according to claim 4, wherein the amino acid residue at position 134 is A, the amino acid residue at position 175 is I, the amino acid residue at position 218 is G, the amino acid residue at position 228 is G, the amino acid residue at position 261 is F and/or the amino acid residue at position 323 is G.

6. Protein or fragment according to claim 1, comprising a hydrophobic amino acid at position 473 and at position 480, wherein said hydrophobic amino acid is selected from the group consisting of valine (V), isoleucine (I), leucine (L) and methionine (M), and wherein the amino acid residue at position 171 is P and the amino acid residue at position 134 is A.

7. Protein or fragment according to claim 6, wherein the amino acid residue at position 38 is P and the amino acid residue at position 40 is G, and/or the amino acid residue at position 175 is I, the amino acid residue at position 218 is G, the amino acid residue at position 261 is F, the amino acid residue at position 228 is G, and/or the amino acid residue at position A323 is G.

8. Protein or fragment according to claim 1, comprising a hydrophobic amino acid at position 473 and at position 480, wherein said hydrophobic amino acid is selected from the group consisting of valine (V), isoleucine (I), leucine (L) and methionine (M), and wherein the amino acid residue at position 171 is P, the amino acid residue at position 170 is P and the amino acid residue at position 44 is P.

9. Protein or fragment according to claim 1, comprising a hydrophobic amino acid at position 473 and at position 480, and wherein the amino acid residue at position 171 is P, the amino acid at position 38 is P, the amino acid residue at position 40 is G, the amino acid residue 134 is A, the amino acid residue at position 218 is G and the amino acid residue at position 228 is G.

10. Protein or fragment according to claim 1, wherein the hydrophobic amino acid at position 473 and the hydrophobic amino acid at position 480 is valine (V).

11. Protein or fragment according to claim 1, comprising a truncated F1 domain, preferably wherein the truncated F1 domain does not comprise the transmembrane and cytoplasmic regions.

12. (canceled)

13. Protein or fragment according to claim 11, wherein the truncated F1 domain comprises the amino acids 113-488 of the HPIV1 F protein.

14. Protein or fragment according to claim 11, wherein a heterologous trimerization domain is linked to the truncated F1 domain.

15. Soluble human parainfluenza virus 1 (HPIV1) F protein or fragment thereof, comprising a truncated F1 domain and an F2 domain, comprising an amino acid sequence of the truncated F1 and F2 domain of an F protein of an HPIV1 strain, wherein the amino acid residue at position 473 and/or 480 is a hydrophobic amino acid, wherein said hydrophobic amino acid is selected from the group consisting of valine (V), isoleucine (I), leucine (L) and methionine (M), and wherein the amino acid residue at position 171 is P, wherein the numbering of the amino acid positions is according to the numbering is amino acid residues in SEQ ID NO: 1.

16. Protein or fragment according to claim 15, wherein the hydrophobic amino acid at position 473 and at position 480 is valine (V).

17. Protein or fragment according to claim 15, wherein the truncated F1 domain does not comprise the transmembrane and cytoplasmic regions preferably, wherein the truncated F1 domain comprises the amino acids 113-488 of the HPIV1 F protein.

18. (canceled)

19. Protein or fragment according to claim 15, wherein the protein does not comprise a heterologous trimerization domain.

20. Protein or fragment according to claim 1, wherein the protein is trimeric.

21. Protein or fragment according to claim 1, comprising an amino acid sequence selected from the group consisting of SEQ ID NO: 4, 6-11, 13-36, 39-41, and 45-48, or an amino acid sequence with at least 90% amino acid sequence identity, or a fragment thereof.

22-27. (canceled)

28. A method for vaccinating a subject against HPIV1, the method comprising administering to the subject a composition according to claim 1.

29. (canceled)

30. (canceled)
